(19) United States
(12) Patent Application Publication
(10) Pub. No.: US 2005/0273070 A1
(43) Pub. Date: Dec. 8, 2005

(54) Absorbent article with low coefficient of friction between materials of differential tensions

(76) Inventors: Christopher P. Olson, Neenah, WI (US); Marianne K. Leick, Appleton, WI (US)

Correspondence Address: DORITY & MANNING, P.A., POST OFFICE BOX 1449, GREENVILLE, SC, 29602-1449, US

(21) Appl. No.: 11/197,599

(22) Filed: Aug. 4, 2005

Related U.S. Application Data
(62) Division of application No. 10/301,881, filed on Nov. 21, 2002.

(51) Int. Cl. A61F 13/15

(52) U.S. Cl. 604/385.22

(57) ABSTRACT

An absorbent article is provided having a chassis with a front waist region, a back waist region, and a crotch region extending between the front and back waist regions. At least one said regions being stretchable in at least one of a transverse direction and a longitudinal direction to render a desired degree of stretchability to the article. For any stretchable region wherein at least two materials are in frictional sliding contact upon the region being stretched, the materials have a coefficient of friction therebetween not greater than about 0.40.

### RELATED APPLICATION

The present application is a Divisional Application of U.S. application Ser. No. 10/301,881 filed on Nov. 21, 2002.

### TECHNICAL FIELD OF THE INVENTION

The present invention relates generally to the field of disposable absorbent articles and garments, such as children's training pants, diapers, incontinence articles, feminine care products, swim pants, diaper pants, and the like, and more particularly to combinations of materials for use in such articles.

### BACKGROUND

Many types of disposable absorbent articles such as disposable diapers, training pants, feminine care articles, incontinence articles, swim pants, diaper pants, and the like, utilize a chassis incorporating different types of materials, including an absorbent body structure. The chassis may include, for example, an absorbent pad and surge layer sandwiched between a bodyside liner and an outer cover member.

From the aspects of product comfort, performance, size range, etc., it is desirable for many types of absorbent articles to incorporate elastomeric materials into various components of the article chassis. For example, it is widely known to incorporate stretchable or elastomeric side panels in a child's training pant article. Such a configuration is known, for example, from the HUGGIES® PULL-UPS® disposable training pants from Kimberly-Clark Corp. of Neenah, Wis. Also, it is a common feature to incorporate full or partial elastic waistband structures in a wide variety of disposable diapers, training pants, and the like. Elastomeric outer covers or bodyside liners are used in different types of absorbent articles.

The “stretchable” features of such articles naturally results in chassis materials frictionally sliding against each other. For example, an elastomeric outer cover member may slide against a generally non-stretchable absorbent pad or surge layer material. Alternately, elastomeric materials having different stretchable tensions may also slide against each other in various article configurations. For example, an elastomeric outer cover member may slide against an elastic coform absorbent pad, or against an elastomeric bodyside liner along the side portions of the chassis. In general, with absorbent articles having “stretchable” features, materials of differing elasticity or stretchability are in sliding frictional contact with each other, or elastomeric materials are in sliding contact with non-elastomeric materials.

The present invention recognizes the potential problems associated with disproportionately high differences in coefficients of friction between sliding materials in absorbent articles, and provides a method and material combinations for addressing such problems.

### SUMMARY OF THE INVENTION

Objects and advantages of the invention will be set forth in part in the following description, or may be obvious from the description, or may be learned through practice of the invention.

For a stretch absorbent article such as a training pant to function as designed, elastomeric materials typically must slide against non-elastomeric materials (or higher stretch tension materials) when the article is stretched. This is particularly true when an elastomeric material is disposed directly against a non-elastomeric material. The concept also applies when different types of elastomeric materials having different stretch tensions are in sliding contact. If one material stretches “easier” than the other material, there will be relative sliding movement between the materials upon stretching the article.

In general, the present Applicants have realized that significant coefficients of friction (“COF”) between materials in sliding contact in a stretch absorbent article may lead to undesirable results. For example, the less elastomeric or non-elastomeric material may inhibit the more elastomeric material from stretching to the extent or ease it was designed to. This may cause problems with respect to product comfort and performance. The Applicants have found that benefits may be obtained by minimizing friction between materials in sliding contact within an absorbent article as a function of the COF between such materials. By limiting the COF between the materials, the article elongates at a lower tension because the inhibition to stretch by frictional forces is minimized. The consumer can more readily feel the elongation at a lower applied tension. Elongation at lower tensions provides the article with a “softer” more comfortable feel against the wearer's skin over a wider range of body sizes. Increased stretch of elastomeric materials results in less material being needed in particular applications and, thus, the more economical use of such materials. Increased elongation at lower tensions also limits the stress put on the article when it is pulled on and worn. This may enable the use of “weaker” and potentially less expensive adhesive or other fastening materials.

The present invention is premised at least in part on the realization just described and provides methods for making absorbent articles wherein the interface coefficients of friction between materials in stretch absorbent articles is minimized through selection of materials and/or various treatments to decrease the interface coefficients of friction. Embodiments of absorbent articles according to the invention will be described in greater detail below.

Additional aspects of the invention will also be described below in greater detail with reference to embodiments shown in the figures.

### BRIEF DESCRIPTION OF THE FIGURES

FIG. 1 is a perspective view of an exemplary absorbent article in accordance with the invention.

FIG. 2 is a perspective view of an alternate embodiment of an absorbent article according to the invention shown in partial cut-away.

FIG. 3 is a bodyfacing plan view of an embodiment of an absorbent article that may incorporate features of the present invention.

FIG. 4 is a cross-cut view of the article shown in FIG. 3 taken along the lines indicated in FIG. 3.

### DETAILED DESCRIPTION

The invention will now be described in detail with reference to particular embodiments thereof. The embodiments are provided by way of explanation of the invention, and are not meant as a limitation of the invention. For example, features described or illustrated as part of one embodiment may be used with another embodiment to yield still a further embodiment. It is intended that the present invention include these and other modifications and variations as come within the scope and spirit of the invention.

Within the context of the present description, the following terms may have the following meanings:

“Attached” and “joined” refers to the bonding, adhering, connecting, and any other method for attaching or joining two elements. Two elements will be considered to be attached or joined together when they are bonded directly to one another or indirectly to one another, such as when each is directly attached to an intermediate element.

“Elastomeric” refers to a material or composite which can be elongated by at least 25% of its relaxed length and which will recover, upon release of the applied force, at least 10% of its elongation. It is generally preferred that the elastomeric material or composite be capable of being elongated by at least 100%, more preferably by at least 300%, of it relaxed length and recover at least 50% of its elongation. An elastomeric material is thus stretchable and “stretchable” and “elastomeric” may be used interchangeably.

“Elastic” or “Elasticized” means that property of a material or composite by virtue of which it tends to recover its original size and shape after removal of a force causing a deformation. An elastic material is thus also elastomeric.

“Neck-bonded” laminate refers to a composite material having an elastic member that is bonded to a non-elastic member while the non-elastomeric member is extended in the machine direction creating a necked material that is elastic in the cross-direction. Examples of neck-bonded laminates are disclosed in U.S. Pat. Nos. 4,965,122; 4,981,747; 5,226,992; and 5,336,545, which are incorporated herein by reference in their entirety for all purposes.

“Stretch-bonded” laminate refers to a composite material having at least two layers in which one layer is a gatherable layer and the other layer is an elastic layer. The layers are joined together when the elastic layer is in an extended condition so that upon relaxing the layers, the gatherable layer is gathered. For example, one elastic member can be bonded to another member while the elastic member is extended at least about 25% of its relaxed length. Such a multiplayer composite elastic material may be stretched until the non-elastic layer is fully extended. Examples of stretch-bonded laminates are disclosed, for example, in U.S. Pat. Nos. 4,720,415, 4,789,699, 4,781,966, 4,657,802, and 4,655,760, which are incorporated herein by reference in their entirety for all purposes.

“Neck-stretch-bonded laminate (NSBL)” as used herein refers to a laminate made from the combination of a neck-bonded laminate and a stretch-bonded laminate. Examples of necked stretch bonded laminates are disclosed in U.S. Pat. Nos. 5,114,781 and 5,116,662, which are incorporated herein in their entirety by reference thereto for all purposes. Of particular advantage, a necked stretch bonded laminate can be stretchable in both the machine and cross-machine directions.

“Nonwoven web” refers a web that has a structure of individual fibers or threads which are interlaid, but not in an identifiable, repeating manner. Nonwoven webs may be formed, for example, by a variety of processes including melt-blowing, spunbonding, and bonded carded web processes.

“Sheet” refers to a layer which may be either a film or a nonwoven web.

“Necked” or “necked material” refers to any material which has been narrowed in at least one dimension by application of a tensioning force.

“Member” when used in the singular can refer to a single element or a plurality of elements.

Coefficients of Friction (“COF”) as used herein are relative values determined from measurements of particular dynamic or kinetic coefficients of friction between material pairs. The COF values may be referred to as relative interface coefficients of friction. The kinetic coefficient of friction values can be determined using a particular variation of ASTM method D 1894-00.

“Frictional sliding contact” as used herein refers to the interface property of adjacent layers in an article that is intended to be stretched in the course of use of the article wherein the layers have different stretch properties. If one layer stretches more or less than the other layer, the relative difference in stretch will result in frictional sliding contact between the layers.

Various aspects and embodiments of the invention will be described in the context of material combinations for disposable absorbent articles, such as disposable diapers, children's training pants, incontinence articles, feminine care products, diaper pants, disposable swim pants, and the like. It should be appreciated that this is for illustrative purposes only, and that the invention is not limited to any particular absorbent article, or absorbent articles in general. The material combinations according to the invention may have beneficial uses in any number of applications, such as protective medical clothing, drapes, gowns, and the like.

The present invention takes into account the realization discussed above and provides a method for making an absorbent disposable article having a desired degree of stretchability in at least one region. The article may have, for example, stretchable sides and/or waist portions, or the entire chassis may be stretchable. Various such embodiments will be discussed in greater detail below. For each region of the absorbent article that is stretchable, it is determined whether any of the materials in the region are in frictional sliding contact with each other upon the article being stretched. The materials are in frictional sliding contact if they are disposed against each other and one material stretches more or less than the other material as the article is stretched. If such sliding materials are present, the interface COF (kinetic) between the materials is determined. If the COF between the materials is greater than a defined maximum value, for example about 0.4, measures are taken to decrease the COF to a value below the defined maximum value. In this way, the highest interface COF between any two materials in the article that are in sliding frictional contact upon the article being stretched is not greater than the defined maximum value.

Applicants have found that a desirable defined maximum COF value is generally not greater than about 0.4, and more desirably less than or equal to about 0.3.

The materials in the stretchable regions may be specifically selected so as to have an inherent interface COF not greater than the defined maximum value. Particular suitable material combinations are set forth below. Alternately, the materials may have an inherent interface COF greater than the defined maximum value and one or both of the materials may be treated with a lubricating substance, such as Teflon® or silicon, to decrease the interface COF. In an alternate embodiment, one or both of the materials may treated with any combination of known treatments to reduce the interface COF. Such treatments may include, for example, chemical, mechanical, thermal, sonic, and electromagnetic treatments. Various methods for carrying out these treatments are well known to those skilled in the art and a detailed explanation thereof is not necessary for an understanding or appreciation of the present invention.

In an alternate embodiment, the materials may have an inherent interface COF greater than the defined maximum value and include a slip member disposed between the materials. The slip member being of a material such that the interface COF's between the slip member and each of the materials is less than the defined maximum value. The slip member may be any manner of material or substance. For example, the slip member may be as simple as a single layer of material, for example a non-elastomeric material.

In an alternate embodiment, the step of taking measures to decrease the COF may include disposing a slip member between the materials. The slip member may be a material sheet or layer, for example a non-elastomeric sheet, selected such that interfaces between the slip member and the respective materials have coefficients of friction below the defined maximum value, for example not greater than about 0.4. The slip member may be any material or substance that essentially functions as a bearing interface between the materials. Any number or variation of materials may be used in this regard.

The present invention also encompasses any manner of absorbent article utilizing the benefits of the material combinations and considerations described herein. An absorbent article according to the invention may have a chassis with a front waist region, a back waist region, and a crotch region extending between the front and back waist regions. At least one of the regions is stretchable, for example in the transverse direction. The region may also be stretchable solely in the longitudinal direction, or in the transverse and longitudinal directions. The article may include multiple such stretchable regions, or the entire chassis may be stretchable. For any of the stretchable regions having at least two materials in frictional sliding contact with each other upon the article being stretched, for example as the article is put on or worn, the interface COF between such materials is generally not greater than a defined maximum value, for example not greater than about 0.40, or not greater than about 0.30.

Exemplary embodiments of absorbent articles will be generally described herein. However, it should be appreciated that the invention is not limited to the described embodiments. The construction and materials used in conventional absorbent articles vary widely and are well known to those of skill in the art. A detailed explanation of every such material and construction is not necessary for purposes of describing the present invention.

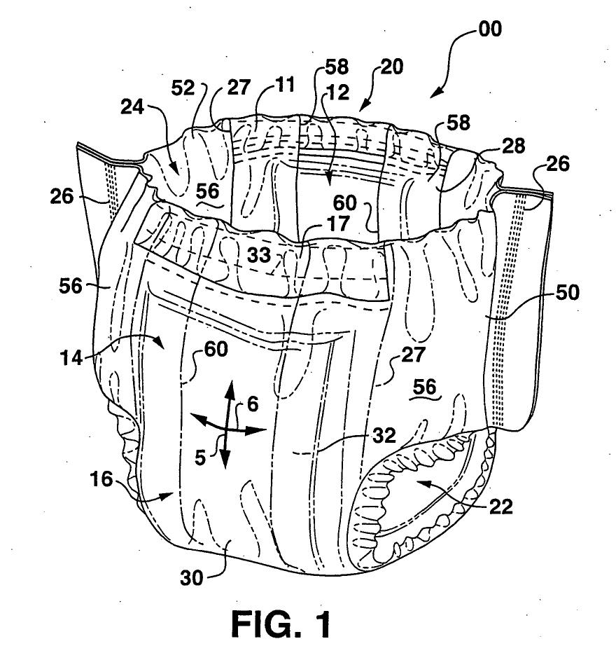

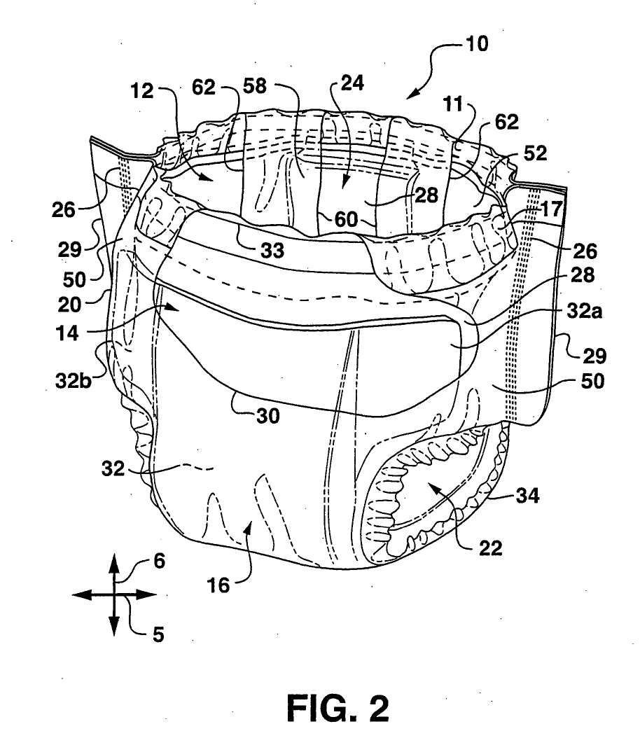

With reference to FIG. 1 in general, an article, such as the representatively shown child's training pant 10, is illustrated. This pant 10 is similar in construction and materials to the HUGGIES® PULL-UPS® disposable training pants from Kimberly-Clark Corp. The article 10 includes a body or chassis 20 having a lengthwise, longitudinal direction 6, a lateral, transverse cross-direction 5, a front waist region 14, a back waist region 12, and an intermediate crotch region 16 interconnecting the front and back waist regions. The waist regions 12 and 14 comprise those portions of the article 10 which when worn, wholly or partially cover or encircle the waist or mid-lower torso of the wearer. In particular configurations, the front 14 and back 12 waist regions may include elastic front and back waistband portions 17, 11 incorporating elastic members 33. In the embodiment of FIG. 1, the elastic waistband portions 11, 17 extend only partially across their respective waist regions, as illustrated in FIG. 2. In an alternate embodiment, the waistband portions 17, 11 may be generally continuous around the waist opening of the article. The waist elastics 33 may be composed of any suitable elastomeric material, such as an elastomeric film, an elastic foam, multiple elastic strands, an elastomeric fabric, and the like. Embodiments of waistband structures that may be utilized with articles 100 according to the invention are also described in U.S. Pat. Nos. 5,601,547; 5,500,063; 5,545,158; 6,358,350 B1; 6,336,921 B1; and 5,711,832, incorporated by reference in their entirety for all purposes.

The intermediate crotch region 16 lies between and interconnects the waist regions 14 and 12, and comprises that portion of the article 10 which, when worn, is positioned between the legs of the wearer and covers the lower torso of the wearer. Thus, the intermediate crotch region 16 is an area where repeated fluid surges typically occur in the training pant or other disposable absorbent article.

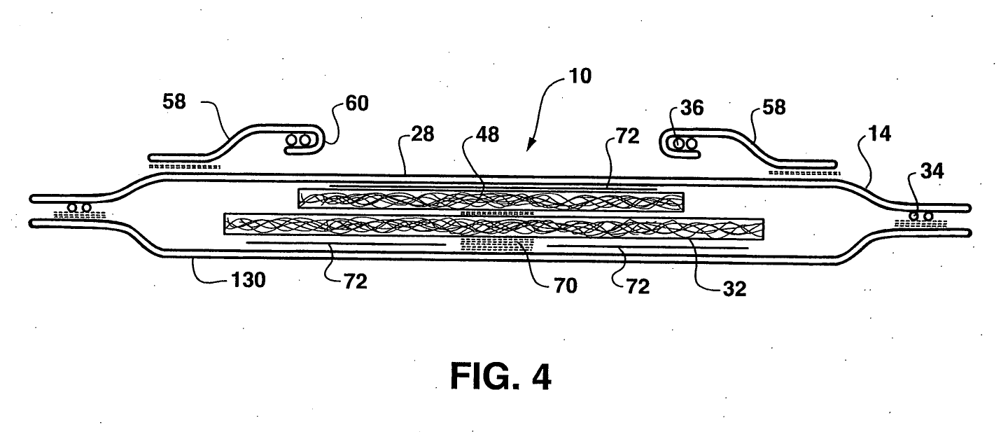

The article 10 includes a substantially liquid-impermeable outer cover member 30, a liquid-permeable bodyside liner 28, and an absorbent body structure 32 sandwiched between the outer cover member 30 and the bodyside liner layer 28. The absorbent body structure may be secured to the outer cover member 30 by an adhesive (such as adhesive 70 in FIG. 4). As described in greater detail below, the absorbent body structure 32 may include a surge layer 48.

Leg elastics 34 may be incorporated along the lateral side margins of the chassis 20 outboard of the absorbent body structure 32 and are configured to draw and hold the chassis 20 against the legs of the wearer. The use of elastic leg members in absorbent articles such as disposable diapers and training pants is widely known and understood in the art.

For various reasons such as product comfort, performance, size range, etc., it is generally known that particular portions and components of the chassis 20 may be formed of elastomeric materials and thus be stretchable, particularly in the lateral or transverse direction 5. In the illustrated embodiment of the article 10 in FIG. 1, the chassis 20 includes stretchable front side panel portions 50 and back side panel portions 52 laterally extending from the central structure of the chassis 20. This configuration is common for training pants and provides the article with a desired degree of stretchability in the transverse direction 5 across the waist regions 12, 14. With a known conventional arrangement as depicted in FIG. 1, the panel portions 50, 52 are defined by generally elastomeric side panels 56 that are attached to the lateral sides of the chassis 20, for example along adhesive seam lines 27. Suitable elastic materials for the side panels 56, as well as a described process of incorporating elastic side panels into a training pant, are described, for example, in the following U.S. Pat. Nos. 4,940,464; 5,224,405; 5,104,116; 5,046,272; and WO 01/88245 all of which are incorporated herein by reference in their entirety for all purposes. In particular embodiments, the elastic material comprises a stretch-thermal laminate (STL), a neck-bonded laminate (NBL), a reversibly necked laminate, a stretch-bonded laminate (SBL) material, or a neck-stretch-bonded laminate (NSBL). Methods of making such materials are described, for example, in U.S. Pat. Nos. 4,663,220; 5,226,992; and the EP Application 0 217 032, all of which are incorporated herein by reference in their entirety for all purposes.

The laterally outboard sides of the panels 56 are joined at side seams 26 to define a pant like structure having a waist opening 24 and leg openings 22. With this type of configuration, the article 10 is pulled on by the wearer in a manner similar to underwear. Particular examples of suitable constructions for securing a pair of elastically stretchable members to the lateral, side portions of an article to extend laterally outward beyond the laterally opposed side regions of the outer cover and liner components of an article can be found in U.S. Pat. No. 4,938,753, which is incorporated by reference herein in its entirety for all purposes.

Desirably, the seams 26 may be separable or tearable so that the pant 10 may be removed from the wearer by tearing or pulling at or along the seams 26 and removing the article in a manner similar to a diaper. In an alternate embodiment, the front and back panel portions 50, 52 may be separable and re-attachable at the side seams 26. A fastening system, such as a hook-and-loop system, may be used to interconnect the waist regions 12 and 14 to define the pant structure and hold the article on a wearer. Additional suitable releasable fastening systems are described in U.S. Pat. No. 6,231,557 B1 and the International Application WO 00/35395, these references being incorporated herein by reference in their entirety for all purposes.

Upon a wearer pulling the article 10 on, and in use of the article 10, the elastomeric side panels exert a tension on the outer cover member 30 and bodyside liner 28 in the transverse direction 5. In embodiments wherein one or both of the liner 28 and outer cover member 30 are elastomeric, either one or both of the materials will be in sliding frictional engagement with a surface of the absorbent body structure 32, for example with a tissue or wrapping layer encasing the absorbent body material. This frictional engagement will be intensified by compression of the absorbent body between the layers. According to the teachings of the present invention, the interface COF between the absorbent body structure 32 and the outer cover member 30 and bodyside liner 28 is below a defined maximum value, for example not greater than about 0.4. This may be accomplished by, for example, selecting the respective materials such that the inherent interface COF is less than the defined maximum limit. A vast number of materials are known to those skilled in the art and a suitable combination of materials may be empirically determined. In an alternate embodiment, a suitable slip member 72 (FIG. 4) may be disposed between the absorbent body structure 32 and outer cover member 30 or bodyside liner 28. In still another embodiment, either or both of the interface surfaces may be treated as described above to decrease the interface COF below the defined maximum value.

In an alternate embodiment of an absorbent article 10 shown in FIG. 2, the panel portions 50, 52 may be extensions of a unitary chassis 20. For example, the panels may be extensions of the outer cover member 30, bodyside liner 28, or both. For example, the chassis 20 may include an elastomeric cover member 30, elastomeric bodyside liner 28, and any combination of other elastomeric components that in combination render a stretchable unitary chassis. The absorbent body structure 32 may include wing portions 32a that extend laterally along the side panels 50, 52 between the outer cover member 30 and bodyside liner 28. In this type of configuration, sliding frictional interfaces may exist between the elastomeric outer cover member 30 and absorbent body structure 32, between the bodyside liner 28 and absorbent body structure 32, and between the outer cover member 30 and bodyside liner 28, for example in areas laterally outboard of the wing portions 32a. The interface COF in these areas may be addressed as discussed above so as not to exceed the defined maximum value.

An article 10 according to the invention may also incorporate longitudinally extending containment flaps 58 disposed over the bodyside liner 28, as generally understood in the art and shown in the Figs. The flaps 58 have longitudinal ends that are attached to the chassis 20 generally at the waistband portions 17, 11. In certain embodiments of the invention, the flaps 58 may comprise separate panels or sheets of material having an outboard lateral side 62 that is attached to the chassis 20 desirably outboard of the underlying absorbent body structure 32. Referring to FIGS. 1 and 2, the flaps 58 may be attached, for example, along the seam line 27. The flaps 58 have a laterally inboard “free” side 60 such that the flaps essentially define a containment pocket along the lateral sides of the absorbent structure 32. The free sides 60 may incorporate flap elastics 36 (FIG. 4) along their longitudinal side, as is generally known in the art. The interface between the flaps 58 and bodyside liner 28 may also be one wherein it is desired that the interface COF be below the defined maximum value. This may be accomplished through selection of materials and/or various treatments as previously described.

The flaps 58 may contain elastic members 36 along at least a portion of their free laterally inward side 60. The construction of such containment flaps 58 is well known and need not be described in detail. Suitable constructions and arrangements for the containment flaps 58 are described, for example, in U.S. Pat. No. 4,704,116, which is incorporated herein by reference for all purposes.

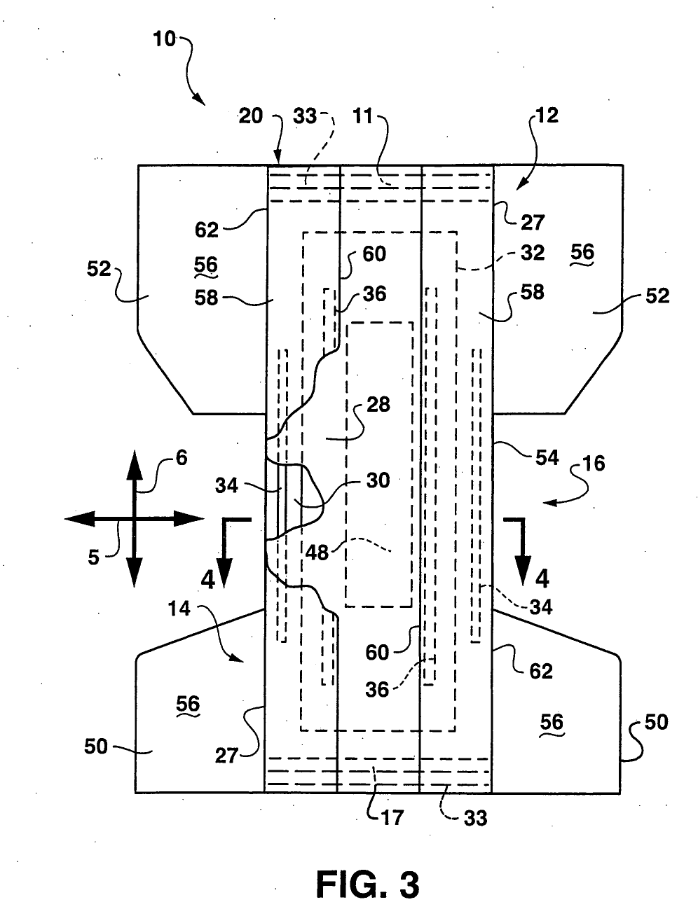

FIG. 3 shows a body facing plan view of a representative article 10, in this case a training pant, in its generally flat-out, uncontracted state (i.e., with substantially all elastic induced gathering and contraction removed). FIG. 4 is a cross-sectional view taken along the lines indicated in FIG. 3. These views particularly illustrate the overlapping relationship and potential sliding frictional engagement between various components of the article. The components are attached or joined together by conventional suitable attachment methods such as adhesive bonds, sonic bonds, thermal bonds, pinning, stitching or any other attachment technique known in the art, as well as combinations thereof. For example, a uniform continuous layer of adhesive, a patterned layer of adhesive, a sprayed pattern of adhesive or an array of separate lines, swirls or spots of construction adhesive may be used to affix the various components.

FIG. 4 depicts the use of slip members 72 disposed between the outer cover member 30 and absorbent body structure 32, and also between the surge layer 48 and bodyside liner 28. As described above, the slip members may be any suitable material having an interface COF with the outer cover member 30, absorbent body structure 32, and/or bodyside liner 28 below the defined maximum value.

Various materials are available and known in the art for use as separate outer cover members 30. Constructions of the outer cover member 30 may comprise a woven or non-woven fibrous web layer which has been totally or partially constructed or treated to impart the desired levels of liquid impermeability to selected regions that are adjacent or proximate the absorbent body. Alternatively, a separate liquid impermeable material could be associated with the absorbent body structure 32. The outer cover member may include a gas-permeable, nonwoven fabric layer laminated to a polymer film layer which may or may not be gas-permeable. Other examples of fibrous, cloth-like outer cover materials can comprise a stretch thinned or stretch thermal laminate material. Although the outer cover member 30 typically provides the outermost layer of the article, optionally the article may include a separate outer cover component member which is additional to the outer cover member.

As mentioned, the outer cover member 30 may be formed substantially from an elastomeric material and may thus be stretchable. The outer cover member 30 may, for example, be composed of a single layer, multiple layers, laminates, spunbond fabrics, films, meltblown fabrics, elastic netting, microporous web, bonded carded webs or foams comprised of elastomeric or polymeric materials. Elastomeric nonwoven laminate webs may include a nonwoven material joined to one or more gatherable nonwoven webs, films, or foams. Stretch bonded laminates (SBL), neck bonded laminates (NBL), and neck stretch bonded laminates (NSBL) are examples of elastomeric composites. Nonwoven fabrics are any web of material which has been formed without the use of textile weaving processes which produce a structure of individual fibers which are interwoven in an identifiable repeating manner. Examples of suitable materials are Spunbond-Meltblown fabrics, Spunbond-Meltblown-Spunbond fabrics, Spunbond fabrics, or laminates of such fabrics with films, foams, or other nonwoven webs. Elastomeric materials may include cast or blown films, foams, or meltblown fabrics composed of polyethylene, polypropylene, or polyolefin copolymers, as well as combinations thereof. The outer cover 130 may include materials that have elastomeric properties obtained through a mechanical process, printing process, heating process, or chemical treatment. For examples such materials may be apertured, creped, neck-stretched, heat activated, embossed, and micro-strained; and may be in the form of films, webs, and laminates.

The absorbent body structure 32 can be any structure or combination of components which are generally compressible, conformable, non-irritating to a wearer's skin, and capable of absorbing and retaining liquids and certain body wastes. For example, the structure 32 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from U.S. Alliance of Childersburg, Ala., USA, and is a bleached, highly absorbent wood pulp containing primarily soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. Methods and apparatus for carrying out such techniques are well known in the art.

As a general rule, the superabsorbent material is present in the absorbent web in an amount of from about 0 to about 90 weight percent based on total weight of the web. The web may have a density within the range of about 0.10 to about 0.35 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 15 times its weight in liquid, and desirably is capable of absorbing more than about 25 times its weight in liquid. Suitable superabsorbent materials are readily available from various suppliers. For example, Favor 880 superabsorbent is available from Stockhausen GmbH of Germany; and Drytech 2035 is available from Dow Chemical Company, of Midland Mich., USA.

After being formed or cut into a desired shape, the absorbent web material may be wrapped or encompassed by a suitable wrap that aids in maintaining the integrity and shape of the absorbent structure 32.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference thereto for all purposes.

The absorbent body structure 32 may include an elastomeric coform absorbent web material. In particular aspects, the elastomeric coform material can have an overall coform basis weight which is at least a minimum of about 50 g/m². The coform basis weight can alternatively be at least about 100 g/m² and can optionally be at least about 200 g/m² to provide improved performance. In addition, the coform basis weight can be not more than about 1200 g/m². Alternatively, the coform basis weight can be not more than about 900 g/m², and optionally, can be not more than about 800 g/m² to provide improved benefits. These values are important because they can provide the absorbent body structure with desired stretchability and structural stability without excessively degrading the physical properties or the liquid-management functionalities of the absorbent body structure. Retention portions having excessively low proportions of elastomeric coform material may not be sufficiently stretchable. An absorbent web material having excessively large amounts of elastomeric coform materials can exhibit an excessive degradation of their absorbency functionalities, such as an excessive degradation of intake, distribution and/or retention properties.

Other examples of elastomeric absorbent structures are described in U.S. Pat. No. 6,362,389 B1, incorporated herein by reference for all purposes.

The absorbent web material utilized in the absorbent body structure 32 is also selected so that the individual absorbent body structure possesses a particular individual total absorbency depending on the intended article of use. For example, for infant care products, the total absorbency can be within the range of about 200-900 grams of 0.9 wt % saline, and can typically be about 500 g of saline. For adult care products, the total absorbency can be within the range of about 400-2000 grams of saline, and can typically be about 1300 g of saline. For feminine care products, the total absorbency can be within the range of about 7-50 grams of menstrual fluid, and can typically be within the range of about 30-40 g of menstrual fluid.

As described, the absorbent body structure 32 may also include a surge management layer 48 which helps to decelerate and diffuse surges or gushes of liquid that may be rapidly introduced into the absorbent body of the article. Desirably, the surge management layer can rapidly accept and temporarily hold the liquid prior to releasing the liquid into the storage or retention portions of the absorbent structure. The surge layer can be located below the bodyside liner layer 28. Alternatively, the surge layer may be located on the body facing surface of the bodyside liner 28. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference in their entirety for all purposes.

### EXAMPLES

For purposes of the present invention, various combinations of materials were tested to determine the kinetic coefficient of friction between such materials. It is believed that the interface kinetic coefficients of friction are reproducible and measurable, and are more representative of actual product use conditions. The kinetic coefficients of friction can be determined using ASTM method D 1894-00. The ASTM procedure is incorporated herein by reference. The ASTM procedure calls for a sample size of 250 mm in the MD and 130 mm in the CD. “Clean” samples of this size typically cannot be “cut” or otherwise obtained from conventional absorbent articles and, thus, to measure coefficients of friction in strict accordance with the ASTM procedure, adequate samples would need to be obtained from the manufacturer or vendor of the materials. To measure the coefficient of friction between samples from an actual absorbent article, it may be necessary to modify the ASTM procedure to accommodate the reduced sample sizes. Such modifications may be readily accomplished by those of ordinary skill in the art.

Various combinations of materials described below were tested. The material samples measured 67 mm in the CD and 152 mm in the MD, and were tested with a modified version of the ASTM D 1894-00 procedure to accommodate the smaller sample size. The samples were tested with a properly calibrated Slip/Peel Tester module SP-101A from Instrumentors, Inc. to determine the dynamic coefficient of friction (COF) between various surfaces of materials.

The materials tested are described below:
SMS
   SMS is Spunbond-melt blown-Spunbond laminate of Montell PF-304 polypropylene
   Total basis weight is 1.0 osy
   Melt blown content is 14%+/−3%
   made in a continuous process set up in the following order: 1 spunbond die bank, 3 meltblown banks in succession, and 1 spunbond bank (5 total banks, 3 layers SB-MB-SB).
   Embossed with a weave pattern per drawing #R-90339-M-024-B/M-015-D
   The SMS is similar to that described in U.S. Pat. No. 4,041,203 and is the SMS used in the outer cover of HUGGIES® LITTLE SWIMMERS® from Kimberly-Clark Corp. of Neenah, Wis., USA
Poly
   White, 0.75 PE film corona treated on both sides, female side wound out
   Embossed pattern
   Identification number is DFST/E XP-414B
   Available from Pliant Corp. of Schaumburg, Ill., USA
Tissue
   16.6 gsm white forming tissue
   Creped wadding
   About 5% moisture content
   Frazier porosity (1 ply) about 300 cfm/sqft
   Available from Cellutissue Corp. of Gouverneur, N.Y., USA
SPunbond
   Either 0.6 or 0.3 osy low denier nonwoven spunbond
   13-23% bond area
   Polypropylene
   Random laid continuous fibers
Necked Stretch Bond Laminate (NSBL)
   0.3 osy facing material of spunbond co-extruded polyethylene/polypropylene necked 60% to 0.5 osy
   Kraton® 6610 elastic film between about 8-15 gsm
Pebax® film
   polyester block amide copolymer film available from ELF Autochem Inc., of Philadelphia, Pa., USA

Results of the tests conducted on combinations of the materials described above are set forth in the following table:

TABLE 1

| | N = 5 | |
|---|---|---|
| | AVG | |
| Material Interface | COF | STD |
| Poly - Spun Bond | 0.34 | 0.03 |
| Poly - tissue | 0.40 | 0.02 |
| NSBL - poly | 0.43 | 0.05 |
| NSBL - SMS | 0.58 | 0.09 |
| NSBL - Tissue | 0.81 | 0.04 |
| NSBL - 0.3 osy SB | 0.97 | 0.06 |
| Pebax - tissue | 1.17 | 0.07 |
| Pebax - Poly | 1.88 | 0.19 |
| Pebax - SMS | 2.10 | 0.25 |
| Pebax - 0.3 osy SB | 2.24 | 0.20 |

Of the material combinations tested, the most desirable stretch material to non-stretch material interface is the NSBL to poly combination. In general, COFs of equal to or less than 0.40 between a stretch and a non-stretch material, or between two stretch materials, are desirable when the objective is to maximize the utilization of the stretch material at minimum tension in an absorbent article chassis.

It should be understood that resort may be had to various other embodiments, modifications, and equivalents to the embodiments of the invention described herein which, after reading the description of the invention herein, may suggest themselves to those skilled in the art without departing from the scope and spirit of the present invention.

1. A method for making an absorbent disposable article having a desired degree of stretchability in at least one region thereof; said method comprising:
for each region of the absorbent article that is stretchable, determine if there are at least two materials in frictional sliding contact with each other upon the article being stretched;
for such materials in frictional sliding contact, determine the coefficient of friction between the materials; and
if the coefficient of friction between the materials is greater than about 0.4, taking measures to decrease the coefficient of friction to not greater than about 0.4 such that a highest coefficient of friction between any two materials in the article that are in sliding frictional contact upon the article being stretched is not greater than about 0.4.

2. The method as in claim 1, wherein said step of taking measures comprises changing at least one of the materials so that an inherent coefficient of friction between the materials is not greater than about 0.4.

3. The method as in claim 1, wherein said step of taking measures comprises treating at least one of the materials with a lubricating substance to decrease the coefficient of friction between the materials to not greater than about 0.4.

4. The method as in claim 1, wherein said step of taking measures comprises disposing a slip member between the materials, the slip member comprising a material such that interfaces between the slip member and the materials have coefficients of friction not greater than about 0.4.

5. An absorbent article made in accordance with the method of claim 1, wherein the absorbent article includes a chassis comprising an absorbent body structure sandwiched between an elastomeric outer cover member and a bodyside liner, said absorbent body structure being less elastomeric than said bodyside liner such that said bodyside liner is in sliding frictional contact against said absorbent body structure with said coefficient of friction therebetween being not greater than about 0.4.

6. The absorbent article as in claim 5, wherein said chassis comprises an absorbent body structure sandwiched between an elastomeric outer cover member and a bodyside liner, said absorbent body structure being less elastomeric than said outer cover member and said bodyside liner such that said outer cover member and said bodyside liner are in sliding frictional contact against said absorbent body structure with said coefficients of friction therebetween being not greater than about 0.4.

7. The absorbent article as in claim 5, wherein said chassis comprises an absorbent body structure sandwiched between an outer cover member and a bodyside liner, said bodyside liner and said outer cover member being generally coextensive and defining side panels of said chassis attached along side seams of said article, said outer cover member and said bodyside liner being in sliding frictional contact along at least a portion of said side panels with said coefficient of friction therebetween being not greater than about 0.4.

8. The absorbent article as in claim 7, wherein at least one of said outer cover member and said bodyside liner comprises an elastomeric material.

9. The absorbent article as in claim 7, wherein said outer cover member and said bodyside liner comprise elastomeric materials.

10. The absorbent article as in claim 7, wherein said absorbent body structure comprises an elastic coform material.

11. The absorbent article as in claim 5, wherein said chassis comprises an absorbent body structure sandwiched between an outer cover member and a bodyside liner, said bodyside liner and said outer cover member being generally coextensive, said outer cover member and said bodyside liner being in sliding frictional contact with said absorbent body with said coefficients of friction between said bodyside liner and said outer cover member relative to said absorbent body structure being not greater than about 0.4.

12. The absorbent article as in claim 11, wherein at least one of said outer cover member and said bodyside liner comprise an elastomeric material.

13. The absorbent article as in claim 11, wherein said outer cover member and said bodyside liner comprise elastomeric materials.

14. The absorbent article as in claim 11, wherein said absorbent body structure comprises an elastomeric coform material.

15. The absorbent article as in claim 5, wherein said materials in relative sliding frictional contact comprise a generally elastomeric material and a generally non-elastomeric material.

16. The absorbent article as in claim 5, wherein said article is one of a disposable diaper, child's training pant, incontinence article, feminine care product, swim pant, and diaper pant.
